(19) United States
(12) Patent Application Publication
(10) Pub. No.: US 2026/0044879 A1
(43) Pub. Date: Feb. 12, 2026

(54) SYSTEMS AND METHODS FOR AUTOMATED PROVISION OF AUXILIARY MEDICATION AND/OR COMPLEMENTARY FOODS DURING PRESCRIPTION MEDICATION PROCESSING

(71) Applicant: Caper Berry, LLC, Chesterfield, MO (US)

(72) Inventors: Jay Baumohl, Chesterfield, MO (US); Douglas D. Rademaker, St. Louis, MO (US)

(21) Appl. No.: 19/207,953

(22) Filed: May 14, 2025

Related U.S. Application Data
(63) Continuation-in-part of application No. 18/800,603, filed on Aug. 12, 2024.

(51) Int. Cl. G06Q 30/0251; G06Q 30/0601; G16H 20/10

(52) U.S. Cl. CPC G06Q 30/0269; G06Q 30/0264; G06Q 30/0633; G16H 20/10

(57) ABSTRACT

Described herein are systems and methods for automated provision of auxiliary medication and/or complementary food during prescription processing. A medication provision computing device is configured to process a prescription for a first medication, the prescription including an identifier of the first medication, and to detect the identifier of the first medication in a stored list of flagged medications. The computing device is further configured to generate, in response to the detecting, provision complementary food provision instructions, the complementary food provision instructions causing a pharmacy computing device to display the complementary food provision instructions for provision of a complementary food to a patient along with the first medication.

### CROSS-REFERENCE TO RELATED APPLICATIONS

This application is a continuation-in-part application of U.S. patent application Ser. No. 18/800,603, filed Sep. 5, 2024, the entire contents of which are hereby incorporated by reference herein in their entirety.

### FIELD

This disclosure relates generally to the field of pharmacy prescription processing and, more specifically, to the automated recommendation and provision of auxiliary medications, commonly referred to as over-the-counter or O-T-C medications, or of one or more complementary foods to be taken with the prescription during the processing of prescription medications at a pharmacy.

### BACKGROUND

It is understood that some prescription medications can affect the levels of specific nutrients, vitamins, and/or minerals in a person's body, as well as the functioning of the body's various systems and sub-systems. It is also understood that some disease states treated by prescription medications can be treated in a complementary manner with auxiliary medications and/or complementary foods. These clinical concepts are referred to as drug-nutrient interactions, nutritional depletion, and/or drug side effects. The person consuming the prescription—that is, the patient—may not be aware of these particular side effects, and therefore may not recognize the benefit of or need to request auxiliary medication or consume the complementary foods. It is also understood that certain medical conditions can benefit from auxiliary medications or complementary foods even without treatment from a prescription medication.

Therefore, there is a need for an electronic prescription processing system that enables real-time, automated provision of one or more auxiliary medications and/or complementary foods during the processing of the prescription medication or a related system for providing auxiliary medicine and complementary recommendations to customers based on their health profile from a retailer shopping/rewards application.

### BRIEF DESCRIPTION

In one aspect, a computer-implemented method for automated provision of auxiliary medication during prescription processing is provided. The method is implemented using a computing device including a processor in communication with a memory. The method includes: (i) processing a prescription for a first medication, the prescription including an identifier of the first medication; (ii) detecting the identifier of the first medication, from the prescription, in a stored list of flagged medications; (iii) in response to the detecting, generating auxiliary medication provision instructions; and (iv) transmitting the auxiliary medication provision instructions to a pharmacy computing device of a pharmacy at which the prescription is being dispensed, the auxiliary medication provision instructions causing the pharmacy computing device to display the auxiliary medication provision instructions for provision of an auxiliary medication to a patient along with the first medication.

In another aspect, a computing device comprising a processor in communication with a memory device storing computer-executable instructions is provided. When the instructions are executed by the processor, the processor is programmed to: (i) process a prescription for a first medication, the prescription including an identifier of the first medication; (ii) detect the identifier of the first medication, from the prescription, in a stored list of flagged medications; (iii) in response to the detecting, generate auxiliary medication provision instructions; and (iv) transmit the auxiliary medication provision instructions to a pharmacy computing device of a pharmacy at which the prescription is being dispensed, the auxiliary medication provision instructions causing the pharmacy computing device to display the auxiliary medication provision instructions for provision of an auxiliary medication to a patient along with the first medication.

In one aspect, a computer-implemented method for automated provision of complementary food during prescription processing is provided. The method is implemented using a computing device including a processor in communication with a memory. The method includes: (i) processing a prescription for a first medication, the prescription including an identifier of the first medication; (ii) detecting the identifier of the first medication, from the prescription, in a stored list of flagged medications; (iii) in response to the detecting, generating complementary food provision instructions; and (iv) transmitting the complementary food provision instructions to a pharmacy computing device of a pharmacy at which the prescription is being dispensed, the complementary food provision instructions causing the pharmacy computing device to display the complementary food provision instructions for provision of a complementary food to a patient along with the first medication. The computer-implemented method may include additional, less, or alternate functionality, including that discussed elsewhere herein.

In another aspect, a computing device comprising a processor in communication with a memory device storing computer-executable instructions is provided. When the instructions are executed by the processor, the processor is programmed to: (i) process a prescription for a first medication, the prescription including an identifier of the first medication; (ii) detect the identifier of the first medication, from the prescription, in a stored list of flagged medications; (iii) in response to the detecting, generate complementary food provision instructions; and (iv) transmit the complementary food provision instructions to a pharmacy computing device of a pharmacy at which the prescription is being dispensed, the complementary food provision instructions causing the pharmacy computing device to display the complementary food provision instructions for provision of a complementary food to a patient along with the first medication. The computing device may include additional, less, or alternate functionality, including that discussed elsewhere herein.

In a further aspect, a computer-implemented method for automated provision of complementary food during prescription processing is provided. The method is implemented using a computing device including a processor in communication with a memory. The method includes: (i) storing a health profile for a first user of a supplementary application; (ii) analyzing the health profile for the first user to detect a first condition including an identifier for the first condition; (iii) detecting the identifier of the first condition in a stored list of flagged conditions; (iii) in response to the detecting, generating complementary food provision instructions; and (iv) transmitting the complementary food provision instructions to be displayed to the first user via the supplementary application, wherein the complementary food instructions cause a user computer device to display the complementary food provision instructions for provision of a complementary food to the first user. The computer-implemented method may include additional, less, or alternate functionality, including that discussed elsewhere herein.

In yet a further aspect, a computing device comprising a processor in communication with a memory device storing computer-executable instructions is provided. When the instructions are executed by the processor, the processor is programmed to: (i) store a health profile for a first user of a supplementary application; (ii) analyze the health profile for the first user to detect a first condition including an identifier for the first condition; (iii) detect the identifier of the first condition in a stored list of flagged conditions; (iii) in response to the detecting, generate complementary food provision instructions; and (iv) transmit the complementary food provision instructions to be displayed to the first user via the supplementary application, wherein the complementary food instructions cause a user computer device to display the complementary food provision instructions for provision of a complementary food to the first user. The computing device may include additional, less, or alternate functionality, including that discussed elsewhere herein.

### BRIEF DESCRIPTION OF THE DRAWINGS

FIGS. 1-6 show example embodiments of methods and systems described herein.

FIG. 1 is a schematic diagram illustrating an example prescription processing system, in accordance with the present disclosure.

FIG. 2 illustrates an example automated auxiliary medication and complementary food provision method that may be implemented using the system shown in FIG. 1.

FIG. 3A illustrates an example decisioning and recording method that may be implemented using the system shown in FIG. 1.

FIG. 3B illustrates an example process for generating incentives that may be implemented using the system shown in FIG. 1.

FIG. 4 illustrates an example prescription update method that may be implemented using the system shown in FIG. 1.

FIG. 5 illustrates an example user computing device that may be used with the system shown in FIG. 1.

FIG. 6 illustrates an example server computing device that may be used with the system shown in FIG. 1.

Like numbers in the Figures indicate the same or functionally similar components.

### DETAILED DESCRIPTION

The present disclosure relates to a prescription processing system that automates provision of auxiliary medications and/or complementary foods during the processing or dispensing of prescriptions, facilitating improved notification of pharmacy employees and patients of the availability and benefits of the auxiliary medication and/or complementary foods. In particular, the prescription processing system includes a medication provision computing device configured to identify prescription medications associated with drug-nutrient interactions, nutritional depletion, or drug side effects, and to automatically initiate the provision of a corresponding auxiliary medication and/or complementary foods upon that identification. In situations where the customer has not waived their rights under HIPAA and the pharmacy cannot share the customer-level prescription data with other internal or third-party information systems, the retailer can generate auxiliary medication and/or or complementary food recommendations based on customer profile data provided in the company's shopping and/or or health and wellness application.

As used herein, an “auxiliary medication” refers to a medication that is not simultaneously prescribed with a prescription issued by a prescriber. Further, auxiliary medications may be specifically associated with certain prescription medications, and may be useful in a) countering negative side-effects of those prescription medications or b) altering the metabolic response of those medications (i.e., absorption, elimination). Auxiliary medication may therefore include over-the-counter (OTC) medication, supplements (e.g., dietary supplements, vitamins, minerals, etc.), probiotics, or combinations thereof. For example, a course of prescribed antibiotics may be known to deplete a patient's body of not only the bacteria causing an illness, but also bacteria beneficial to the patient's gastrointestinal system. In such an example, an auxiliary medication may include a probiotic. As another example, auxiliary medications may include CoQ10 and Vitamin D when a prescribed medication is a statin.

As used herein, a “complementary food” refers to a food item or items that is/are known to be beneficial when taking specific prescriptions and/or during different stages of a disease. Further, complementary foods may be specifically associated with certain prescription medications or generally associated with specific disease states, and may be useful in a) countering negative side-effects of those prescription medications, b) altering the metabolic response of those medications (i.e., absorption, elimination), or c) improving one's health status in general. Complementary food may therefore include fresh or packaged foods. For example, a prescription to treat hyperthyroidism may include a suggestion for non-iodized salt. In another example, a prescription for warfarin may include suggestions for different foods rich in Vitamin K, such as, but not limited to, asparagus, broccoli, brussels sprouts, collard greens, kale, mustard greens, seaweed, spinach, Swiss chard, and/or turnip greens.

The “provision” of a medication or food, such as an auxiliary medication or complementary food, may include a variety of actions to make the patient aware of the auxiliary medication and/or complementary food and present an opportunity for the patient to purchase or otherwise acquire the auxiliary medication and/or complementary food. Provision may include the physical provision of the auxiliary medication and/or complementary food to the patient, as well the identification of the auxiliary medication and/or complementary food to the patient either by printed message(s), message(s) displayed on an electronic display device, or a verbal message (e.g., a recommendation) from a pharmacy employee to the patient. Provision may also include personalized recommendations in the shopping/rewards application made available to customers. Additionally, printed or electronic messages may include promotions, such as coupons, related to the auxiliary medication and/or complementary food.

The prescription processing system of the present disclosure may facilitate improved patient awareness and overall health outcomes and increase non-pharmacy revenues for the retailer on account of prescription processing activity. These benefits may be achieved without significant infrastructure changes or the dedication of significant computing resources. Specifically, the methods of the present disclosure may be implemented in conjunction with existing prescription dispensing systems, enabling rapid adoption across medical and pharmacy systems. The technical problems addressed by this system include at least one of: (i) lack of readily available information regarding medications associated with drug-nutrient interaction or nutrient depletion, both for pharmacy employees and patients; (ii) lack of any automated messaging to the pharmacy employee or the patient regarding the availability or desirability of auxiliary medications and/or complementary foods; and (iii) inability of pharmacies or other entities to track patient purchase or use of auxiliary medications and/or complementary foods.

The methods and systems described herein may be implemented using computer programming or engineering techniques including computer software, firmware, hardware, or any combination or subset thereof, wherein the technical effects may be achieved by: (a) processing a prescription for a first medication, the prescription including an identifier of the first medication; (b) detecting the identifier of the first medication, from the prescription, in a stored list of flagged medications; (c) in response to the detecting, generating provision instructions for auxiliary medication and/or complementary food; and (d) transmitting the provision instructions for auxiliary medication and/or complementary food to a pharmacy computing device of a pharmacy at which the prescription is being dispensed, the provision instructions for auxiliary medication and/or complementary food causing the pharmacy computing device to display the auxiliary medication provision instructions for provision of an auxiliary medication and/or complementary food to a patient along with the first medication.

The resulting technical benefits achieved by this system include at least one of: (i) automated messaging at a pharmacy computing device for recommending or providing auxiliary medications and/or complementary foods; (ii) efficient and up-to-date storage of prescription medication relationships, indications, and counter-indications with auxiliary medications and/or complementary foods; and (iii) improved tracking of patient offers and uptake. At least some of these technical benefits are achieved based on the unique location of the medication provision computing device within the overall prescription processing computing environment. Specifically, the communicative coupling of the medication provision computing device with a pharmacy computing device, as described herein, enables the prescription processing system to respond in real-time to prescription dispensing that may benefit from the provision of an auxiliary medication and/or a complementary food. These advantages are also realized without significant infrastructure changes.

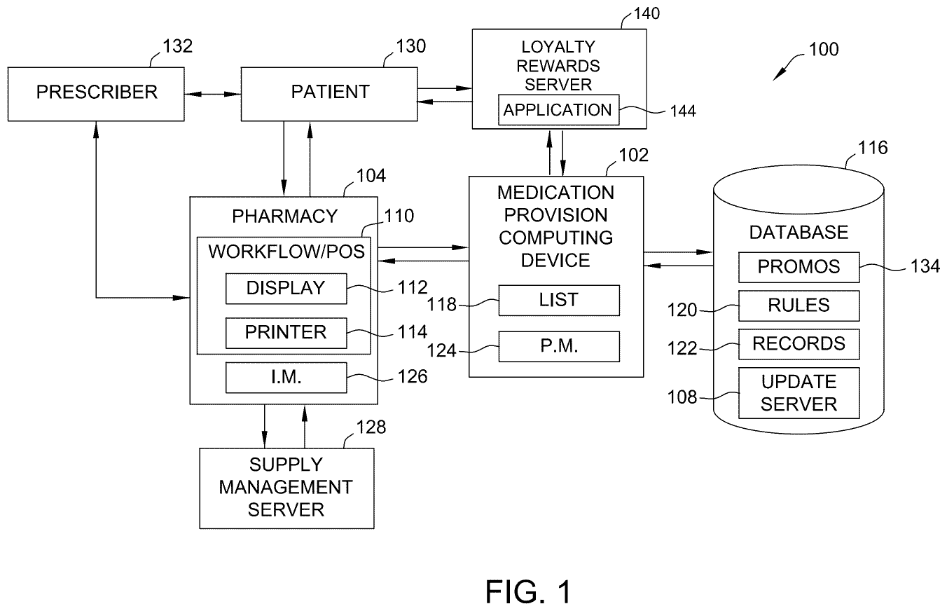

FIG. 1 includes a schematic diagram illustrating an example prescription processing system 100 for automated provision of auxiliary medication and complementary foods during prescription dispensing. In the example embodiment, the prescription processing system 100 includes a medication provision computing device 102, which facilitates at least a portion of the auxiliary medication provision methods and/or the complementary foods provision methods described further herein. The medication provision computing device 102 may include any suitable computing device(s), such as one or more personal computing devices (e.g., laptop computing device, desktop computing device, etc.) server computing device(s), databases, cloud-based computing and/or storage systems, and/or any other device(s). The medication provision computing device 102 enables real-time identification and provision of auxiliary medications and/or complementary foods during the processing and dispensing of a prescription at a pharmacy location 104. “Real-time” refers to processes or routines that occur without substantial delay, such as within seconds or minutes.

In the exemplary embodiment, the medication provision computing device 102 is configured to store a list 118 of flagged medications. This list 118 identifies prescription medications that are associated with recognized side effects for which an auxiliary medication and/or complementary foods may be medically beneficial or recommended. In particular, the list 118 of flagged medications includes prescription medications recognized as causing drug-nutrient interaction and/or nutritional depletion, which can be counteracted by an auxiliary medication and/or complementary food. The list 118 of flagged medications also identifies the auxiliary medication(s) and/or the complementary food(s) with recognized associations to the prescription medication. In some embodiments, the list 118 includes a plurality of entries associating product codes of prescription medications with respective product codes for auxiliary medications, including OTC auxiliary medications, and for complementary foods. These product codes may include, for example but without limitation, National Drug Codes (NDC), Universal Product Codes (UPC), or Generic Product Identifiers (GPI). In some embodiments, the list 118 of flagged medications includes prescription medications recognized as causing other side effects, such as pain, nausea, sleep problems, etc. In some such embodiments, the associated auxiliary medications may include a pain reliever, an anti-nausea medication, a sleep aid, etc. In these embodiments, the associated complementary foods may include foods with omega-3 fatty acids, plain crackers and bananas, and foods rich in tryptophan, etc. That is, although the present disclosure is primarily directed to prescription medications causing drug-nutrient interaction and/or nutritional depletion and to associated auxiliary medications and/or complementary foods to counteract the drug-nutrient interaction and/or nutritional depletion, various other prescription medications, auxiliary medications, and/or complementary foods are also within the scope of the disclosed embodiments. Additionally, where the term “list” is used herein, it should be recognized that the particular data structure is not limited to a one-dimensional array, but may include a table or other two-dimensional array, a self-referential table, or any other suitable data structure.

In the example embodiment, the prescription processing system 100 further includes at least one database 116. The database 116 may be any data storage device and may store, for example, the list 118, rules 120, records 122, and/or any other data described herein. The medication provision computing device 102 is communicatively coupled to the database 116, such that the medication provision computing device 102 can store data in and access data from the database 116 (e.g., using database queries or commands).

As noted, the database 116 of the exemplary embodiment is configured to store one or more rules 120, which control—at least in part—the output(s) from medication provision computing device 102, when the medication provision computing device 102 detects that a prescription for a medication on the list 118 is being dispensed. The list 118 and/or the rules 120 may be transmitted to or stored by the medication provision computing device 102. In some other embodiments, the medication provision computing device 102 generates and stores one or more rules 120 based on the list 118.

Additionally, the database 116 of the exemplary embodiment stores one or more records 122. The records 122 reflect any messaging provided to pharmacy employees and/or patients, and may be associated with a specific prescription claim. The records 122 may also reflect an outcome of the messaging, such as whether a patient was offered and either declined or purchased or otherwise obtained a provided auxiliary medication and/or complementary food.

The prescription processing system 100 further includes a plurality of pharmacies 104. It should be understood that although only one pharmacy 104 is shown, the functionality of the prescription processing system 100, particularly of the medication provision computing device 102, is accessible to any number of pharmacies that are in networked communication with the medication provision computing device 102, as described further herein. Each pharmacy 104 includes at least one pharmacy workflow and point-of-sale (computing) device 110 physically located thereat, referred to generally herein as a pharmacy computing device 110. Other pharmacy computing device 110 may include computing devices (e.g., laptop computing devices, desktop computing devices, tablets, etc.) that are not themselves computing devices but that are physically located at the pharmacy 104 and in networked communication with at least one pharmacy computing device 110. In the example embodiment, the pharmacy computing device 110 includes a display 112 (e.g., one or more screens) and a printer 114. The display 112 or the printer 114 may not be an integral component to the pharmacy computing device 110 but may be communicatively coupled thereto.

In some embodiments, the pharmacy computing device 110 and the medication provision computing device 102 are not physically separate computing devices. For example, the medication provision computing device 102 represents a processing module and/or storage medium including computer-executable instructions, within the processing/memory components of the pharmacy computing device 110, that control the pharmacy computing device 110 to perform the functions described herein as being performed by the medication provision computing device 102. Therefore, the description of various steps performed by the prescription processing system 100 may be applicable to actions executed by the pharmacy computing device 110 and/or the medication provision computing device 102.

Additionally, the prescription processing system 100 includes an update server 108 communicatively coupled to the medication provision computing device 102. In the illustrated embodiment, the database 116 includes the update server 108. The update server 108 may include one or more server computing devices associated with one or more entities that (i) identify recognized associations between prescription medications, auxiliary medications, and complementary foods, (ii) define rules (e.g., rules 120) for the provision of auxiliary medications and complementary foods, (iii) define messaging instructions related to the provision of auxiliary medications and complementary foods, and/or (iv) define promotions applicable to auxiliary medications and complementary foods being provided. For example, the update server 108 may be related to one or more pharmacies (e.g., pharmacies 104), pharmaceutical wholesalers, wholesale distributors, and the like. The update server 108 may transmit update instructions to the medication provision computing device 102, causing the medication provision computing device 102 to update the list 118 of flagged medications and/or the rules 120. The update server 108 may also receive information from the medication provision computing device 102, such as stored records 122. (reference prior comment regarding update server 108). It is understood that in some suitable embodiments, the update server 108 can be omitted.

In some embodiments, the database 116 may also be accessible to one or more other parties, such as a pharmacy 104, pharmaceutical wholesalers, wholesale distributors, and the like. In some of these embodiments, the pharmacy 104 may be associated with a grocery store or other similar retailer. The database 116 may further store data regarding promotions 134 that are applicable to one or more auxiliary medications and/or complementary foods. Each promotion 134 has a promotional offer, that is, what the financial offer related to the purchase of a product is. A promotional offer may include an absolute amount by which a product is discounted (e.g., $1.00), a percentage of a total price by which a product is discounted (e.g., 25%), a discount having specific purchase conditions (e.g., “buy one, get one 50% off”), or any other promotional offer. Additionally, each promotion 134 has a brand identifier and product identifier, identifying the product to which the promotional offer is applied as well as the brand thereof. The product, in the example embodiment, is an auxiliary medication or complementary food. The brand may be an in-house brand (e.g., a brand related to the particular pharmacy 104 or associated grocery store), a generic brand, or a “name brand.”

In some embodiments, the database 116 stores the promotions 134 indexed according to the product identifier, which may be a product code for the auxiliary medication or complementary food. In particular, the product identifier may be the same product code associated with the auxiliary medication or complementary food that is stored within the list 118. In such cases, a promotion 134 can be readily accessed using only the product code that is returned when the list 118 is queried.

In some embodiments, the database 116 stores the promotions 134 including additional details, such a date or date range during which the promotion 134 is active. Additionally, the promotion 134 may include a “weighting” or “ranking” factor. In these embodiments, the database 116 may store multiple promotions 134 related to a same auxiliary medication or complementary food. These promotions 134 may be ranked or weighted such that one promotion 134 is preferred over the others. When that first promotion 134 is inapplicable to a particular transaction (e.g., that particular brand of auxiliary medication or complementary food is unavailable), a second (lower-ranked) promotion 134 may be activated.

The prescription processing system 100 further includes at least one supply management server 128. In the example embodiment, the supply management server 128 is configured to monitor an inventory of pharmacy 104 (or associated grocery store) and manage orders for further inventory, such as when the stock of one or more items is low. The supply management server 128 may be a server computing device that communicates with the pharmacy 104 (e.g., with the pharmacy computing device 110) and the medication provision computing device 102. In some embodiments, the supply management server 128 is a part of the pharmacy computing device 110. For example, the medication provision computing device 102 represents a processing module and/or storage medium including computer-executable instructions, within the processing/memory components of the pharmacy computing device 110, that control the pharmacy computing device 110 to perform the functions described herein as being performed by the supply management server 128. Therefore, the description of various steps performed by the prescription processing system 100 may be applicable to actions executed by the pharmacy computing device 110 and/or the supply management server 128.

The medication provision computing device 102 may further include executable modules such as a promotion manager 124. The promotion manager 124 is configured to identify at least one promotion (e.g., of stored promotions 134) applicable to the purchase of an auxiliary medication (i.e., a provisioned auxiliary medication) or complementary food (i.e., a provisioned complementary food). The promotion manager 124, executed by the medication provision computing device 102, pushes the applicable promotion to the pharmacy computing device 110 during the prescription dispensing process, as described in greater detail herein.

In the exemplary embodiment, the pharmacy 104 (e.g., with the pharmacy computing device 110) includes an inventory manager 126 configured to determine whether an auxiliary medication or complementary food being provisioned is available at the pharmacy 104 or associated grocery store. The inventory manager 126 may communicate with the medication provision computing device 102 and/or the supply management server 128 to determine the availability. When the availability of the product is low (e.g., the number of units falls outside of a threshold) or the product is unavailable at the pharmacy 104 or associated grocery store, the inventory manager 126 may query the supply management server 128 for the availability of a replacement product or similar product. Additionally or alternatively, the inventory manager 126 may communicate with the supply management server 128 to initiate the purchase of one or more products.

At least some communication channels and/or data exchange described herein, such as between various components of the prescription processing system 100, may be implemented using an application programming interface (API) communication ecosystem to enable direct data integration between those components. Additionally, certain forms available to facilitate this data connection may be implemented using standardized form types (e.g., a JAVASCRIPT library) to enable direct integration.

In operation of the prescription processing system 100, a patient 130 is prescribed a first medication by a prescriber 132. The prescription for the first medication may include an identifier of the first medication (e.g., a chemical name, a generic name, a brand name, etc.) and a date the first medication was prescribed, as well as dosage instructions. The prescription is provided to the pharmacy 104 to be dispensed. In some instances, the prescriber 132 transmits the prescription to the pharmacy 104 electronically; in other instances, the patient 130 is provided with a paper prescription that the patient 130 then physically brings to the pharmacy 104 for dispensing.

At the pharmacy 104, the prescription is processed. In some instances, the prescription undergoes preliminary processing or intake processing at the pharmacy computing device 110. The intake processing includes the necessary processing steps to initiate an insurance claim and queue the prescription for filling and dispensing. In the example embodiment, filling of a prescription refers to the various steps taken at the pharmacy 104 to prepare the physical medication that was prescribed, including, for example (but not limited to) the counting of medication into containers, placing medication containers into packaging (e.g., a paper or plastic bag), labeling medication containers and packaging, and the like. Dispensing, by contrast, refers specifically to the process of the patient 130 (or a representative thereof) physically obtaining the prescribed medication (that is, the filled prescription) from the pharmacy 104. Dispensing may include various checkout or purchase steps performed at the pharmacy 104 and may be referred to herein as a dispensing transaction or dispensing process.

The patient 130 is notified (e.g., by a pharmacy employee or the pharmacy computing device 110) that their prescription has been filled and, therefore, is ready for dispensing. The patient 130 (or a representative thereof) arrives at the pharmacy 104 to physically retrieve the prescription medication and, thereby, have the prescription dispensed. In many instances, the patient 130 interacts with a pharmacy employee operating the pharmacy computing device 110. Generally, the pharmacy employee uses the pharmacy computing device 110 to retrieve information about the status of the prescription (e.g., filled, on hold, etc.) and, when the prescription has been filled, to identify the location of the filled prescription within the pharmacy 104. The pharmacy employee locates the filled prescription and, commonly, returns to the pharmacy computing device 110 to initiate a checkout process. In many cases, the pharmacy employee uses the pharmacy computing device 110 to input a code or identifier of the filled prescription to initiate the checkout process, such as by manually inputting the identifier using an input device such as a keyboard, scanning a code using barcode scanner, and the like.

In the example embodiment, the pharmacy computing device 110 transmits the prescription to the medication provision computing device 102 when the checkout process is initiated (e.g., as part of the prescription dispensing process). That is, the functionality of the medication provision computing device 102 may be activated when a dispensing transaction is initiated. In other embodiments, the pharmacy computing device 110 may transmit a prescription to the medication provision computing device 102 during the intake processing, after the intake processing, before filling the prescription, after filling the prescription, or at any other point during intake, filling, or dispensing of the prescription.

In some instances, transmitting the prescription includes transmitting the code or identifier of the prescription to the medication provision computing device 102. The medication provision computing device 102 accesses details of the prescription using this code or identifier, such as from a memory device (e.g., an internal memory device or the database 116). In other instances, the pharmacy computing device 110 may transmit the details of the prescription directly to the medication provision computing device 102.

The medication provision computing device 102 receives the prescription from the pharmacy computing device 110 and processes the prescription as part of the dispensing transaction. As described above, this processing may include retrieving details of the prescription from a memory device or another computing device, or may include processing details of the prescription as received from the pharmacy computing device 110. In particular, the medication provision computing device 102 parses the details of the prescription for an identifier of the first medication for which the prescription was generated. The medication provision computing device 102 may access and process other details of the prescription, such as a date the prescription was generated or issued by the prescriber 132, dosage information, etc.

The medication provision computing device 102 accesses the list 118 of flagged medications. In the example embodiment, the medication provision computing device 102 has received the list 118 at some prior point in time, such as from the update server 108, and has stored the list 118 within an internal memory device or an otherwise accessible memory location, such as the database 116. The medication provision computing device 102 compares the identifier of the first medication to the list 118 to determine whether the first medication is a flagged medication. That is, the medication provision computing device 102 determines whether the first medication is associated with a recognized indication, such as drug-nutrient interaction, nutrient depletion, or drug side effect. In some embodiments, this comparison is embodied as a database query of a memory of the medication provision computing device 102 (or the database 116), using the identifier of the first medication.

When the first medication is not on the list 118 of flagged medications, the query may return no results or may be considered unsuccessful. In such instances, the medication provision computing device 102 may take no further action with respect to that prescription. Alternatively, the medication provision computing device 102 transmits a null response to the pharmacy computing device 110, representing that the first medication is not flagged, and the dispensing transaction proceeds as normal.

A successful query returns a “hit” or a match to the input identifier of the first medication, indicating that the first medication is a flagged medication. The query also returns the respective identifier of any auxiliary medication(s) and/or complementary food(s) associated with the (identified) medication. In some embodiments, the identifier of an auxiliary medication and/or complementary food is a product code. When the medication provision computing device 102 detects the identifier of the first medication within the list 118 of flagged medications (e.g., in response to a successful database query), the medication provision computing device 102 initiates one or more processes to provide the auxiliary medication and/or complementary food represented by the (returned) identifier. In the event that the patient profile within the 110 computing device indicates that the patient would have an allergic reaction to the auxiliary medication and/or complementary food, the auxiliary medication and/or complementary food would not be offered.

In the example embodiment, the medication provision computing device 102 compares the identifier of the auxiliary medication and/or complementary food to the rules 120 to determine whether a rule to provision the auxiliary medication or complementary food exists. This comparison may be embodied as a database query, similar to the query described above. For example, when there is no rule 120 about the provision of the auxiliary medication or complementary food, the query may return no results or may be considered unsuccessful. In such instances, the medication provision computing device 102 may take no further action with respect to that prescription. Alternatively, the medication provision computing device 102 transmits a null response to the pharmacy computing device 110, representing that there is no rule 120 to provide the auxiliary medication or complementary food, and the dispensing transaction proceeds as normal.

A successful query returns a “hit” or a match to the input identifier of the auxiliary medication and/or complementary food, indicating that there exists a rule 120 for the provision of the auxiliary medication and/or complementary food. The query also returns the respective rule(s) 120. In some embodiments, the rule 120 is structured as computer-executable instructions that control the medication provision computing device 102 to generate and transmit provision instructions for the auxiliary medication and/or complementary food to the pharmacy computing device 110. The provision instructions for the auxiliary medication and/or complementary food may be generated by the medication provision computing device 102 as computer-executable instructions that control subsequent actions of the pharmacy computing device 110, as described further herein. That is, these instructions, when processed by the respective computing device, cause that computing device to perform functions defined within the instructions. In some embodiments, as described further herein, a returned rule 120 may also include computer-executable instructions that control the medication provision computing device 102 to execute the promotion manager 124.

In the above description, the querying of the list 118 and the rules 120 are described as separate functions performed by the medication provision computing device 102 during the dispensing transaction. In other embodiments, the list 118 and the rules 120 may be stored in a same memory location (e.g., of a database or other memory integral to or accessible by the medication provision computing device 102). In such embodiments, a single database query may be sufficient to return both (i) any identifier of an auxiliary medication and/or complementary food associated with the prescription medication, from the list 118, and (ii) any rules 120 for the provision of that auxiliary medication and/or complementary food.

The medication provision computing device 102 is configured to initiate any actions defined in the retrieved rule 120. In the exemplary embodiment, the medication provision computing device 102 transmits the auxiliary medication provision instructions and/or the complementary food provision instructions to the pharmacy computing device 110. This transmission, and subsequent processing of the provision instructions for the auxiliary medication and/or complementary food, causes the pharmacy computing device 110 to generate a message. The message includes an identifier of the auxiliary medication or complementary food and, in the example embodiment, a description of the association between the prescribed medication and the auxiliary medication or complementary food. For example, where the prescribed medication is an antibiotic, the message may identify a probiotic medication or probiotic foods based on the type of antibiotic and an explanation that the antibiotic may cause undesirable effects related to the patient's gastrointestinal system, and therefore the probiotic medication or probiotic food is recommended (or provided) to counteract this side effect. The message may further include instructions directing the pharmacy employee to recommend, retrieve, or otherwise provide the auxiliary medication or probiotic food to the patient 130. The provision instructions for the auxiliary medication and/or complementary food also cause the pharmacy computing device 110 to enable display of this message to the pharmacy employee and/or to the patient 130.

More specifically, in some embodiments, the provision instructions for the auxiliary medication and/or complementary food cause the pharmacy computing device 110 to activate printer 114 to initiate printing of the message. The printer 114 may print the message as part of or as a supplement to other printed materials commonly provided during prescription dispensing, such as instructional materials, medication identification materials, a receipt, and the like, collectively referred to as a “package insert.” The message or the package insert may additionally include information regarding dosage information, other drug interactions, allergy information, and the like.

Additionally or alternatively to activating the printer 114, the provision instructions for the auxiliary medication and/or complementary food cause the pharmacy computing device 110 to display the message on the display 112. The display 112 may include a display (e.g., a screen) facing the pharmacy employee, and the pharmacy computing device 110 causes the display 112 to display the message as a pop-up or other virtual overlay on the interface otherwise active on the display 112.

When the message is printed in conjunction with the package insert, the message is made apparent or obvious to the pharmacy employee, who is responsible for providing the package insert to the patient 130 with the dispensed prescription. Additionally or alternatively, when the message is displayed as an overlay on the display 112, the message is made apparent or obvious to the pharmacy employee, who engages with the pharmacy computing device 110 (and, therefore, the display 112) to complete the prescription dispensing. In this way, the automatic processes performed by the medication provision computing device 102 and the pharmacy computing device 110, as described herein, produce an “interrupter” in the steps taken by the pharmacy employee, which improves the likelihood that (i) the pharmacy employee takes notice of the message and, thereby, (ii) the pharmacy employee provides the auxiliary medication and/or complementary food information to the patient 130. Alternatively, in some embodiments, the printer 114 prints the message separately from the package insert; however, such a separately printed message may also serve as an interrupter and therefore provide the above benefits.

Additionally or alternatively, in some embodiments, the display 112 includes a display (e.g., a screen) facing the patient 130, and the pharmacy computing device 110 causes the display 112 to display the message as a pop-up or other virtual overlay on the interface otherwise active on the display 112. In some embodiments, the computer-executable instructions cause the pharmacy computing device 110 (or the medication provision computing device 102) to transmit the message to a computing device of the patient, such as within an email, SMS message, push notification, etc. In these embodiments, the message is made apparent or obvious to the patient 130, which improves the likelihood that the patient 130 will read the message and request the auxiliary medication and/or complementary food be provided to them.

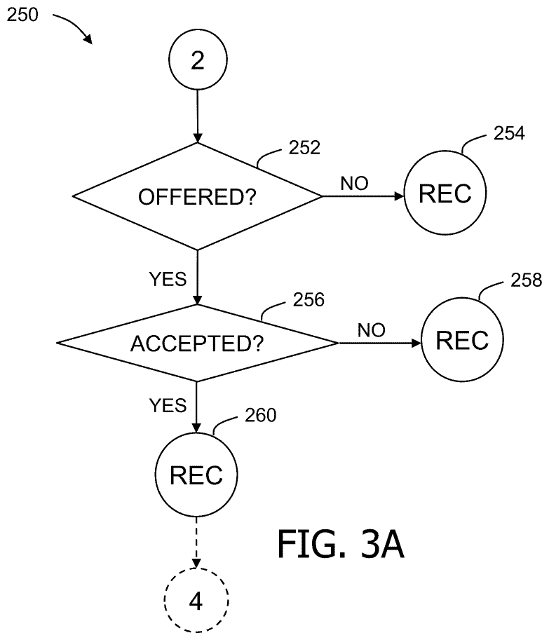

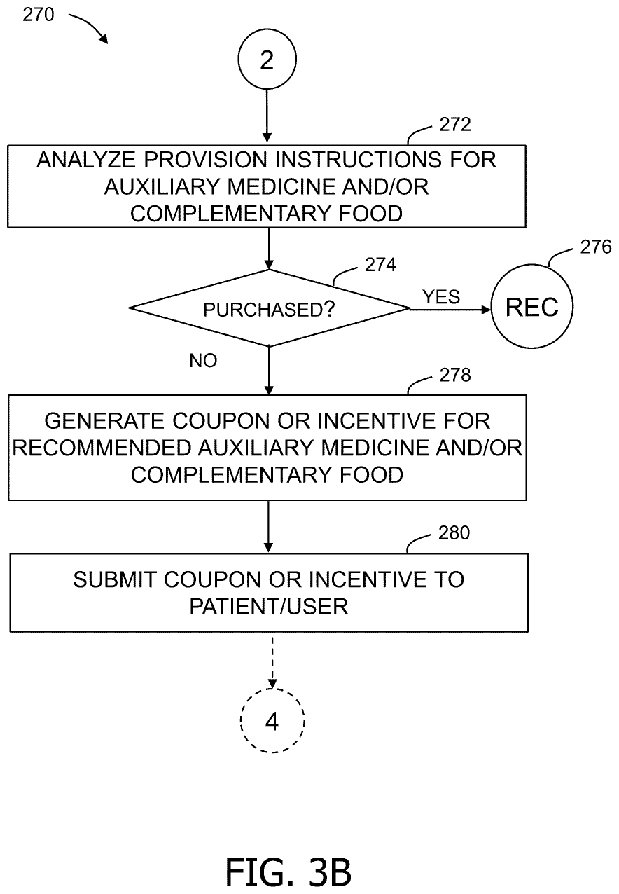

In response to the message, the pharmacy employee may provide the auxiliary medication and/or complementary food to the patient 130. As described above and in more detail with respect to FIG. 3A, provision of the auxiliary medication and/or complementary food may include making a verbal recommendation to the patient 130, directing the patient 130 to the location of the auxiliary medication and/or complementary food, and/or physically retrieving the auxiliary medication and/or complementary food for presentation to the patient 130. The patient 130 decides whether to obtain the auxiliary medication and/or complementary food concurrently or in conjunction with the filled prescription, as part of the dispensing transaction.

In certain embodiments, the medication provision computing device 102 may use the date of the prescription to determine whether to transmit the provision instructions for the auxiliary medication and/or complementary food to the pharmacy computing device 110. In one example embodiment, the retrieved rule 120, associated with the first medication identified in the prescription, defines a required currency of the prescription. The medication provision computing device 102 compares the date of the prescription to a current date (i.e., a date on which the prescription is being dispensed). If the amount of time between the date of the prescription and the current date falls outside of a threshold date range in the rule 120, the medication provision computing device 102 may not transmit provision instructions for the auxiliary medication and/or complementary food to the pharmacy computing device 110. The promotion manager can be configured to generate offers upon subsequent refills of the prescription or some other interval.

In some embodiments, the rule 120 retrieved by the medication provision computing device 102 includes instructions for the medication provision computing device 102 to determine whether any promotion is applicable to the ongoing dispensing transaction. In response, the medication provision computing device 102 activates the promotion manager 124. The promotion manager 124 queries the database 116 (or another memory device, such an internal memory of the medication provision computing device) with the identifier of the auxiliary medication and/or complementary food, which may include a product code.

When there is no promotion 134 applicable to the auxiliary medication and/or complementary food, the query may return no results. In such instances, the medication provision computing device 102 may proceed with the dispensing transaction as described above. A successful query returns a “hit” or a match to the input identifier of the auxiliary medication and/or complementary food, indicating that there exists a promotion 134 applicable to the auxiliary medication and/or complementary food. The query also returns the respective promotion 134.

The promotion manager 124 parses the promotion 134 for certain details, to ensure the promotion is relevant or applicable to the ongoing dispensing transaction. For instance, the promotion manager 124 compares a current date to a date or date range during which the promotion 134 is active. In some embodiments, the promotion manager 124 then causes the promotion 134 to be sent to the pharmacy computing device 110 (e.g., as part of the provision instructions for the auxiliary medication and/or complementary food), which is instructed to display the promotion 134 concurrently with (or after) the message, described above.

In some embodiments, after confirming the promotion 134 is applicable, the promotion manager 124 parses the promotion 134 for the brand identifier and, if different from the input identifier for the auxiliary medication and/or complementary food, the product identifier. The promotion manager 124 activates the inventory manager 126 to determine whether the specific product identified in the promotion 134 is available at the pharmacy 104. The inventory manager 126 uses the brand identifier and product identifier, parsed or otherwise available from the promotion manager 124, to query the supply management server 128.

The supply management server 128 returns, to the inventory manager 126, an indication of the availability of particularly branded auxiliary medication or complementary food at the pharmacy 104 or associated grocery store. This indication may be a binary data element (e.g., “true,” when the product is available, “false,” when the product is unavailable) or a value representing a number of units or percentage of total availability.

When the particularly branded auxiliary medication or complementary food is available, the promotion manager 124 then causes the provision instructions for the auxiliary medication and/or complementary food to include the promotion 134, as described above (causes the generation and transmission of separate promotion provision instructions). When the particularly branded auxiliary medication or complementary food is not available, the promotion manager 124 may cause the medication provision computing device 102 to proceed with the dispensing transaction as described above, without any promotion 134. Alternatively, the promotion manager 124 re-queries the database 116 to request a promotion 134 for similar or related product, or a different promotion 134 for a different brand of the same product. If the re-query is successful, the promotion manager 124 may repeat the process described above to confirm availability.

In some embodiments, when the particularly branded auxiliary medication or complementary food is not available, the inventory manager 126 directs the supply management server 128 to order one or more units of the product. The inventory manager 126 may return, to the promotion manager 124, details of the order. The promotion manager 124 may then include the order details in the instructions sent to the pharmacy computing device 110. The pharmacy computing device 110 displays or otherwise communicates the order details to the pharmacy employee and/or the patient 130, such that the patient 130 is advised when the product will again be available for purchase, and, in some cases, whether the promotion 134 will still apply. The pharmacy computing device 110 may prompt the patient 130 to pre-order the auxiliary medication or complementary food during the current dispensing transaction to ensure the promotion 134 will be applied, such as by displaying a prompt on one or more displays 112. In still further embodiments, the inventory manager 126 communicates with the supply management server 128 to initiate an order of the product to the patient's home (or other preferred delivery address).

In some embodiments, the promotion 134 is structured as computer-executable instructions that control the medication provision computing device 102 to generate the auxiliary medication provision instructions to include promotional details. The auxiliary medication provision instructions may be generated by the medication provision computing device 102 as computer-executable instructions that control subsequent actions of the pharmacy computing device 110. That is, these instructions, when processed by the respective computing device, cause that computing device to perform functions defined within the instructions.

Therefore, in some embodiments, the message displayed via the pharmacy computing device 110 further includes the promotion 134 associated with the auxiliary medication or complementary food. The promotion 134 may be embodied as a physical or digital coupon. In some embodiments, the provision instructions for the auxiliary medication and/or complementary food-generated by the medication provision computing device 102 and transmitted to the pharmacy computing device 110—further cause the pharmacy computing device 110 to activate the printer 114 to initiate printing of the promotion 134, such as in the form of a physical coupon. In some embodiments, the provision instructions for the auxiliary medication and/or complementary food further cause the pharmacy computing device 110 to display the promotion 134 on one or more displays 112, as a digital coupon. Additionally or alternative, the pharmacy computing device 110 transmits the promotion 134 as a digital coupon to a computing device of the patient 130, such as via email, SMS, push notification, personalized online advertisements, social media, online landing pages, digital wallet, etc. In some further embodiments, the pharmacy computing device 110 is directly or indirectly in communication with a store shopping application for the pharmacy 104 or an associated retail store, such as loyalty rewards application 144. In the embodiment wherein the store shopping application 144 is for the pharmacy 105, the pharmacy computing device 110 directly instructs the store shopping application 144 to display the digital coupon to the patient 130. In embodiments where the store shopping application 144 and the pharmacy 105 are separate, the digital coupons are transmitted to the store shopping application and/or the pharmacy shopping application 144. In some further embodiments, the digital coupons are provided to the store or pharmacy shopping application 144 along with reward points that the patient 130 would receive for purchasing the auxiliary medication and/or complementary food. In still further embodiments, the digital coupons are printed and mailed to the address associated with the patient 130.

The promotion 134 identifies the auxiliary medication and/or complementary food and the related details, such as the promotional offer, the product, and the brand, to purchase or otherwise obtain the auxiliary medication and/or complementary food concurrently with the prescription medication (i.e., during a same dispensing transaction). The patient 130 may then choose whether to employ the promotional 134 to receive the auxiliary medication and/or complementary food concurrently with the prescription medication.

The medication provision computing device 102 is configured to generate and store records 122 of the dispensed prescriptions associated with flagged medications. The records 122 include the identifier of the flagged medication and various other details, such as a time/date of prescription dispensing, an identifier of the particular pharmacy employee who initiated the prescription dispensing, whether a promotion 134 was available and/or applied, and the like. The medication provision computing device 102 may generate a record 122 in conjunction with the transmission of the provision instructions for the auxiliary medication and/or complementary food.

As described further herein with respect to FIG. 3A, in some embodiments, the medication provision computing device 102 receives an indication of whether the patient 130 was offered an identified auxiliary medication and/or complementary food (e.g., in the form of a displayed or printed message, a verbal recommendation from a pharmacy employee, etc.), and, in response, whether the patient 130 actually obtained the auxiliary medication and/or complementary food (e.g., accepted a recommendation, purchased an auxiliary medication and/or complementary food physically presented to them by the pharmacy employee, etc.). For example, upon the termination of a transaction associated with the prescription dispensing, the pharmacy computing device 110 transmits a message including the identifier of the dispensed prescription and a binary data element that is either true-indicating acceptance of the auxiliary medication and/or complementary food by the patient—or false—indicating no such acceptance. The medication provision computing device 102 updates a stored record 122 having the same prescription identifier, for example, with the binary data element.

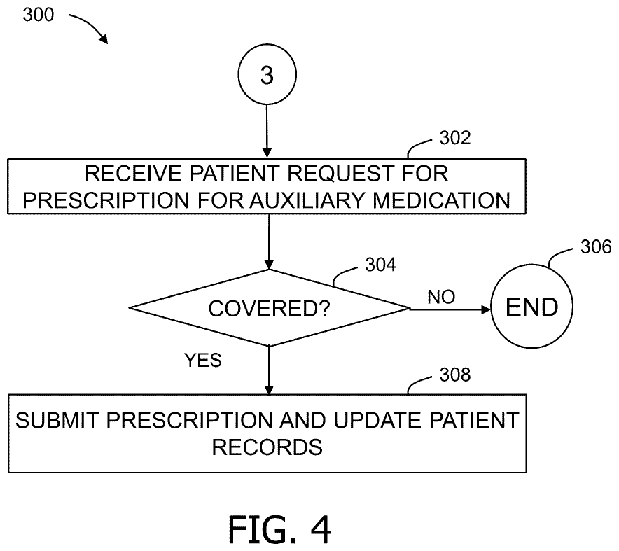

As described further herein with respect to FIG. 4, in some embodiments, the patient 130 accepted and obtained the auxiliary medication and/or complementary food according to the processes described above. However, the patient 130 may prefer to have the auxiliary medication and/or complementary food be prescribed, for example, by the prescriber 132. For example, the patient 130 may have a particular insurance or other policy coverage that would partially or fully reimburse the patient 130 for the cost of the auxiliary medication and/or complementary food. In such cases, the pharmacy employee may perform conventional steps to have the prescription requested and may update a file or record for the patient 130. The medication provision computing device 102 may, in some instances, update a stored record 122 to reflect that the auxiliary medication and/or complementary food is now prescribed to the patient 130 and, therefore, should no longer be provided as described herein.

The medication provision computing device 102 therefore maintains a robust data set that can be leveraged to make conclusions and analyze trends related to the auxiliary medications and/or complementary foods. For example, the pharmacy 104 may leverage the records 122 to assess the relative acceptance of various auxiliary medications and/or complementary foods. Additionally or alternatively, the success of particular pharmacy employees in providing the auxiliary medications and/or complementary foods may also be assessed. The records 122 may be accessible to parties within the prescription processing system 100, for example, via a web-accessible portal enabled by an API connection, or by some other software platform. The data may be made available to certain parties, including entities outside of the prescription processing system 100, in an aggregated or other anonymized format, such that no personally identifiable or otherwise sensitive information related to patient(s) 130 is vulnerable.

The medication provision computing device 102 may periodically receive update messages from the update server 108. The messages may include an updated list of flagged medications, updated rules 120, and the like. The medication provision computing device 102 is configured to process the update message and update any stored data in accordance with the update message.

In some further embodiments, the patient 130 or other user may access a loyalty rewards application 144 on a loyalty rewards server 140. The application 144 may be a website, a shopping application, and/or a pharmacy shopping application to access information about the auxiliary medications and/or complementary foods for specific prescription medications. The loyalty rewards application 144 provides recommendations for the auxiliary medications and/or complementary foods based upon the prescription drug selected for look-up from one or more reference databases. The loyalty rewards application 144 may also provide digital coupons for the purchase of one or more of the auxiliary medications and/or complementary foods. The reference database would be available to potential patients 130, as well as pharmacists and pharmacy technicians to answer patient and other customer questions.

In these further embodiments, the medication provision computer device 102 is in communication with the loyalty rewards application 144 to provide personalized recommendations in the corresponding application. In some embodiments, the medication provision computing device 102 determines the auxiliary medications and/or complementary foods to recommend and provides those recommendations to the loyalty rewards application 144 to generate incentives and/or coupons to provide to the patient 130.

In other embodiments, the patient 130 has generated a healthcare profile in the loyalty rewards application 144. In these embodiments, the loyalty rewards application 144 provides a series of questions to the patient 130 for the patient 130 to respond to. For example, the questions may be about the patient's health and/or their healthcare goals, such as eating healthier, losing weight, improved cardiac health, and/or dealing with chemotherapy. The loyalty rewards application 144 generates a healthcare profile for the patient 130 that is used to generate specific recommendations and/or incentives for the patient 130. In these embodiments, the pharmacy 104 may encourage the patient 130 to create the healthcare profile.

In still further embodiments, the pharmacy 104 provides the option to share healthcare information with the loyalty rewards application 144 to the patient 130. In these embodiments, the patient 130 may opt-in to sharing at least some of their healthcare information with the loyalty rewards application 144. The patient 130 may select categories and/or specific healthcare details to share with the loyalty rewards application 144. The loyalty rewards application 144 then makes specific recommendations to the patient 130 based upon the information provided to it.

While FIG. 1 shows the loyalty rewards server 140 in communication with the medication provision computing device 102, in other embodiments, the loyalty rewards server 140 is in communication with the pharmacy computing device 110 instead.

In additional embodiments, the loyalty rewards server 140 is in communication with one or more servers associated with the pharmacy 104 and/or grocery store. When the patient 130 enters their health information into the loyalty rewards application 144, application 144 determines one or more complementary foods to recommend. These recommendations are generated by an algorithm. In some embodiments, the algorithm recommends categories of auxiliary medications and/or or complimentary foods to the patient 130. In further embodiments, the application 144 provides information to the retailer that may assist them in establishing a promotional campaign. In these embodiments, the application 144 provides information about categories of complementary food and/or auxiliary medications that are being recommended, as well as numbers of users that these items have been recommended to. In these embodiments, the application 144 only provides numbers of users rather than any personally identifiable information. Then the promotions may be transmitted to all users. In other embodiments, the application 144 matches individual uses to different promotions based upon their entered health profiles.

In some further embodiments, the loyalty rewards server 140 stores a health profile for a first user 130 of a supplementary application, wherein the supplementary program is a loyalty rewards application 144. The loyalty rewards server 140 analyzes the health profile for the first user 130 to detect a first condition including an identifier for the first condition. The first condition may be a health condition, such as low iron, a disease state, such as Stage I diabetes, or other condition, such as age group, gender, or other factor. The loyalty rewards server 140 detects the identifier of the first condition in a stored list of flagged conditions. In response to the detecting, the loyalty rewards server 140 generates complementary food provision instructions. Then the loyalty rewards server 140 transmit the complementary food provision instructions to be displayed to the first user 130 via the supplementary application 144. The complementary food instructions cause a user computer device 400 to display the complementary food provision instructions for provision of a complementary food to the first user 130.

In some further embodiments, the loyalty rewards server 140 displays, via the supplemental application 144, an instructional message related to the provision of the complementary food. In some further embodiments, the complementary food provision instructions further cause the supplemental application 144 to display a promotion 134 associated with the complementary food. In some further embodiments, the loyalty rewards server 140 cause the printing of a promotion 144 associated with the complementary food to be sent to the first user 130. In other embodiments, the loyalty rewards server 140 generates an electronic transmission of a promotion 144 associated with the complementary food to be transmitted to the first user 130.

In some further embodiments, the loyalty rewards server 140 determines whether the complementary food instructions were provided to the first user 130. The loyalty rewards server 140 determines whether the first user 130 purchased the complementary food. When the complementary food was provided and purchased, the loyalty rewards server 140 generates an acceptance record identifying the complementary food.

In some further embodiments, the loyalty rewards server 140 generates a list of flagged conditions. The list of flagged conditions including a plurality of entries associating product codes of respective product codes of complementary foods. The loyalty rewards server 140 stores the list of flagged conditions in the memory, such as database 116, to establish the stored list of flagged conditions.

In some further embodiments, the loyalty rewards server 140 determines an availability of the complementary food. When the availability is at or below a threshold, the loyalty rewards server 140 causes display of a prompt to submit an order for the complementary food, such as one the pharmacy computer device 110.

In some further embodiments, the loyalty rewards server 140 receives a plurality of health information from the first user 130, such as via the application 144. The loyalty rewards server 140 generates the health profile for the first user 130 using the plurality of health information from the first user 130.

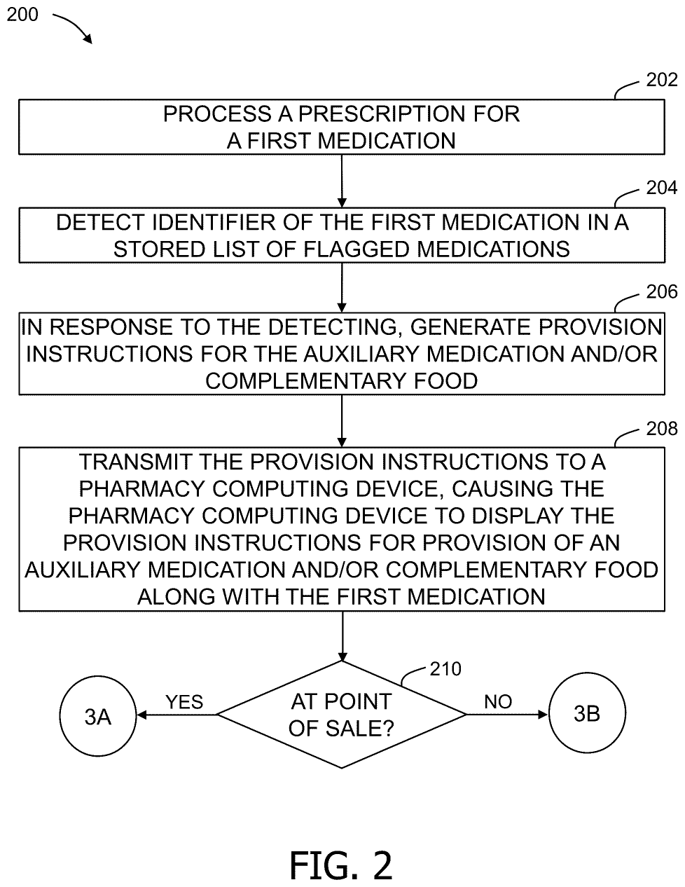

FIG. 2 is an example flow diagram illustrating a method 200 for automated provision of auxiliary medication and/or complementary foods during prescription dispensing. The method 200 may be implemented using one or more components of the prescription processing system 100, including the medication provision computing device 102 (both shown in FIG. 1).

The method 200 includes, in the example embodiment, processing 202 a prescription for a first medication. The prescription includes an identifier of the first medication. The method 200 also includes detecting 204 the identifier of the first medication, from the prescription, in a stored list of flagged medications. The method 200 also includes generating 206, in response to the detecting, provision instructions for the auxiliary medication and/or complementary food, and transmitting 208 the provision instructions for the auxiliary medication and/or complementary food to a pharmacy computing device of a pharmacy at which the prescription is being dispensed. The provision instructions for the auxiliary medication and/or complementary food cause the pharmacy computing device to display the provision instructions for the auxiliary medication and/or complementary food for provision of an auxiliary medication and/or complementary food to a patient along with the first medication.

In some embodiments, the pharmacy computing device 110 includes a pharmacy point-of-sale device. The method 200 further includes, in the example embodiment, determining 210 whether or not the patient is at the point of sale for the pharmacy 104. If yes, then the method proceeds to process 250, shown in FIG. 3A. If not, then the method proceeds to process 270, shown in FIG. 3B.

The method 200 may include additional, fewer, or alternative steps, in other embodiments. For example, in some embodiments, the pharmacy computing device includes a pharmacy point-of-sale device, and the transmitting 208 causes the pharmacy point-of-sale device to initiate printing of an instructional message related to the provision of the auxiliary medication and/or complementary food. In some such embodiments, the transmitting 208 further causes the pharmacy point-of-sale device to initiate printing or electronic transmission of a promotion associated with the auxiliary medication and/or complementary food.

In some embodiments, the pharmacy computing device includes a pharmacy point-of-sale device, and the transmitting 208 causes the pharmacy point-of-sale device to display an instructional message related to the provision of the auxiliary medication and/or complementary food on a display of the pharmacy point-of-sale device. In some such embodiments, the transmitting 208 further causes the pharmacy point-of-sale device to initiate printing or electronic transmission of a promotion associated with the auxiliary medication and/or complementary food.

In some embodiments, the prescription further includes a date at which the prescription was generated by a prescriber. The method 200 may further include: (i) parsing the prescription for the date; (ii) comparing the date of the prescription to a current date; and (iii) only transmitting (208) the provision instructions for the auxiliary medication and/or complementary food when the current date is within a threshold date range relative to the date of the prescription.

In some embodiments, the method 200 also includes determining whether the auxiliary medication and/or complementary food was offered to the patient, determining whether the auxiliary medication and/or complementary food was accepted by the patient, and, when the auxiliary medication and/or complementary food was offered and accepted, generating an acceptance record identifying the first prescription and the auxiliary medication and/or complementary food.

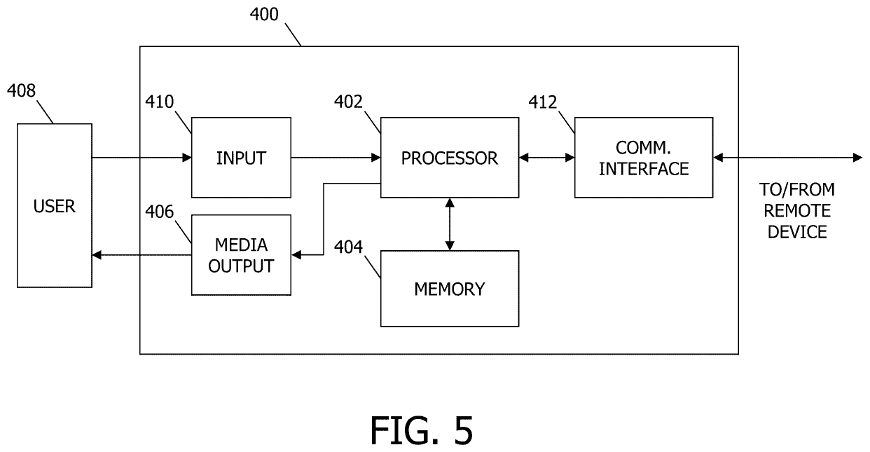

In some embodiments, the method 200 further includes generating the list of flagged medications. The list of flagged medications includes a plurality of entries associating product codes of prescription medications with respective product codes of OTC auxiliary medications and/or complementary foods. The method 200 may further include storing the list of flagged medications (e.g., in a memory device, such as the database 116, shown in FIG. 1, or the memory 404, shown in FIG. 5) to establish the stored list of flagged medications.

In some embodiments, the method 200 further includes determining an availability of the auxiliary medication and/or complementary food, and, when the availability is at or below a threshold, causing display of a prompt to submit an order for the auxiliary medication and/or complementary food.

In some embodiments, the method 200 proceeds with an additional process 250, shown in FIG. 3A. Specifically, the auxiliary medication and/or complementary food is provisioned as described above. The provision is embodied as an offer, which may include a verbal recommendation, printed offer, and/or digital offer. The patient 130 may then accept or decline the auxiliary medication and/or complementary food.

In some embodiments, the pharmacy computing device 110 is configured to detect and record whether the auxiliary medication and/or complementary food was offered 252. The pharmacy computing device 110 may automatically record this information, such as when a message is displayed or printed. Additionally or alternatively, the pharmacy computing device 110 may prompt the patient 130 or the pharmacy employee to confirm whether the auxiliary medication and/or complementary food was offered 252. The pharmacy computing device 110 is further configured to detect and record whether the auxiliary medication and/or complementary food was accepted 256. The pharmacy computing device 110 may automatically record this information, such as by parsing transaction details of the completed dispensing transaction. Additionally or alternatively, the pharmacy computing device 110 may prompt the patient 130 or the pharmacy employee to confirm whether the patient accepted (and obtained) 256 the auxiliary medication and/or complementary food. The pharmacy computing device 110 is further configured to transmit any of the above details to the medication provision computing device 102, whether in real-time during the dispensing transaction, as a dataset after the dispensing transaction is complete, and/or within a batch of datasets periodically (e.g., once a day, once an hour, etc.).

The medication provision computing device 102 is configured to identify, from the received data, whether the auxiliary medication and/or complementary food was offered 252. In some instances, the response is a binary data element (e.g., “true,” when the auxiliary medication and/or complementary food was offered, “false,” when the auxiliary medication and/or complementary food was not offered). If the patient 130 was not offered (252) the auxiliary medication and/or complementary food, the medication provision computing device 102 records 254 the result. Specifically, the medication provision computing device 102 may generate a new record 122 or update an existing record 122 to reflect the auxiliary medication and/or complementary food was not offered during the dispensing transaction.

When the auxiliary medication and/or complementary food was offered 252, the medication provision computing device 102 may continue to parse and review the received data. Specifically, the medication provision computing device 102 is further configured to identify, from the received data, whether the auxiliary medication and/or complementary food was accepted 256. In some instances, the response is a binary data element (e.g., “true,” when the auxiliary medication and/or complementary food was accepted/obtained, “false,” when the auxiliary medication was not accepted/obtained). If the patient 130 did not accept (256) the auxiliary medication and/or complementary food, the medication provision computing device 102 records 258 the result. Specifically, the medication provision computing device 102 may generate a new record 122 or update an existing record 122 to reflect the auxiliary medication and/or complementary food was not accepted during the dispensing transaction.

When the auxiliary medication and/or complementary food was both offered 252 and accepted 256, the medication provision computing device 102 records 260 the result. Specifically, the medication provision computing device 102 may generate a new record 122 or update an existing record 122 to reflect the auxiliary medication and/or complementary food was offered and accepted during the dispensing transaction. In still other embodiments, the method 200 proceeds with an additional process 300, shown in FIG. 4.

FIG. 3B illustrates an example process 270 for generating incentives that may be implemented using the system 100, shown in FIG. 1. In some embodiments, the method 270 includes analyzing 272 the provision instructions for the auxiliary medications and/or complementary foods. This analysis 272 may include determining other medications and foods that may assist the patient 130. The analysis 272 may further include determining categories, SKUs (stock keeping units). In addition, the analysis 272 may also include determining 274 whether or not the patient 130 has purchased the auxiliary medications and/or complementary foods, such as within a certain number of days before and/or after the prescription was prescribed and/or picked up.

If the determination 274 is that the auxiliary medication and/or complementary food was purchased by the patient 130, the method 270 includes recording 276 that interaction. If the determination 274 is that the auxiliary medication and/or complementary food has not been purchased, the method 270 includes generating a coupon or incentive to recommend the auxiliary medicine and/or complementary food. The method 270 also includes submitting 280 the coupon or incentive to the patient 130. In some embodiments, the coupon is submitted 280 to the patient 130 via the loyalty rewards application 144. In other embodiments, a physical copy of the coupon or incentive is printed and mail to an address of the patient 130.

In still other embodiments, the method 200 proceeds with an additional process 300, shown in FIG. 4. Specifically, in some instances, a patient 130 requests a prescription for the auxiliary medication and/or complementary food. The pharmacy computing device 110 receives 302 the patient request (e.g., based on input from a pharmacy employee). In some cases, the pharmacy computing device 110 is configured to perform conventional prescription claim processing on the auxiliary medication and/or complementary food, to determine 304 whether the prescription would be covered by an insurance policy or other medical coverage of the patient 130. If not, the pharmacy computing device 110 and the medication provision computing device 102 may take no further action with respect to that dispensing transaction, and the method 200 ends 306 as illustrated in FIG. 4. In another suitable embodiment, the pharmacy computing device 110 and the medication provision computing device 102 may take further action with respect to that dispensing transaction. For example, the method 200 can include offering the auxiliary medication and/or complementary food to the patient such as the offering process 250 illustrated in FIG. 3A. In such an embodiment, the method 200 continues and does not end at the ends step 306.

If the pharmacy computing device 110 determines 304 l that the prescription for the auxiliary medication and/or complementary food would be covered, the pharmacy computing device 110 may perform conventional prescription processing, such as submitting 308 the prescription for approval (e.g., by the prescriber 132) and updating records associated with the patient 130. In some embodiments, the pharmacy computing device 110 transmits an indication to the medication provision computing device 102 that the auxiliary medication and/or complementary food is prescribed and, in the future, therefore, should not be provided according to the methods described herein. The pharmacy computing device 110 and/or the medication provision computing device 102 update one or more records, such as records 122, with a flag. When such a flag is detected, in future dispensing transactions, the auxiliary medication and/or complementary food will not be provided.

FIG. 5 illustrates an example configuration of a user system 400, such as the medication provision computing device 102 or the pharmacy computing device 110 (both shown in FIG. 1). In the example embodiment, the user system 400 includes a processor 402 for executing instructions. In some embodiments, executable instructions are stored in a memory area 404. The processor 402 may include one or more processing units, for example, a multi-core configuration. The memory area 404 is any device allowing information such as executable instructions and/or written works to be stored and retrieved. The memory area 404 may include one or more computer readable media.

The user system 400 also includes at least one media output component 406 for presenting information to a user 408 (e.g., the patient 130, the prescriber 132, a pharmacy employee at the pharmacy 104, etc.). The media output component 406 is any component capable of conveying information to the user 408. In some embodiments, the media output component 406 includes an output adapter such as a video adapter and/or an audio adapter. An output adapter is operatively coupled to the processor 402 and operatively connectable to an output device such as a display device, a liquid crystal display (LCD), organic light emitting diode (OLED) display, or “electronic ink” display, or an audio output device, a speaker or headphones. The media output component 406—or another output component of the user system 400 (not shown) may include a printer or other output device configured to provide a physical media output from the user system 400.

In some embodiments, the user system 400 includes an input device 410 for receiving input from the user 408. The input device 410 may include, for example, a keyboard, a pointing device, a mouse, a stylus, a touch sensitive panel, a touch pad, a touch screen, a gyroscope, an accelerometer, a position detector, or an audio input device. A single component such as a touch screen may function as both an output device of the media output component 406 and the input device 410. The user system 400 may also include a communication interface 412, which is communicatively connectable to a remote device (e.g., the medication provision computing device 102, the pharmacy computing device 110, the update server 108, all shown in FIG. 1). The communication interface 412 may include, for example, a wired or wireless network adapter or a wireless data transceiver for use with a mobile phone network, Global System for Mobile communications (GSM), 3G, 4G, 5G, or other mobile data network or Worldwide Interoperability for Microwave Access (WIMAX).

Stored in the memory area 404 are, for example, computer readable instructions for providing a user interface to the user 408 via the media output component 406 and receiving and processing user input from the input device 410. A user interface may include, among other possibilities, a web browser and client application (“app”).

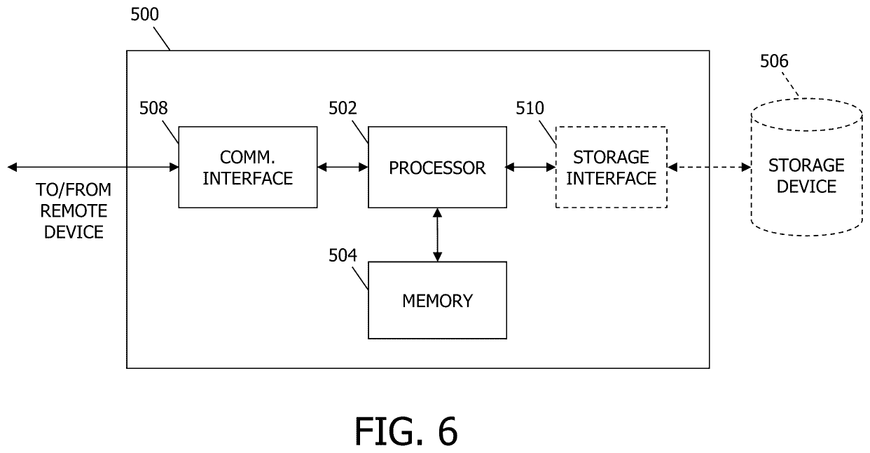

FIG. 6 shows an example configuration of a server system 500. The server system 500 may include, but is not limited to, the medication provision computing device 102, the update server 108, the supply management server 128 (all shown in FIG. 1), and/or computing device(s) associated with any party to the prescription processing system 100 (also shown in FIG. 1).

The server system 500 includes a processor 502 for executing instructions. Instructions may be stored in a memory area 504, for example. The processor 502 may include one or more processing units (e.g., in a multi-core configuration) for executing instructions. The instructions may be executed within a variety of different operating systems on the server system 500, such as UNIX, LINUX, Microsoft Windows®, etc. More specifically, the instructions may cause various data manipulations on data stored in the memory 504 and/or in a storage device 506 (e.g., create, read, update, and delete procedures). It should also be appreciated that upon initiation of a computer-based method, various instructions may be executed during initialization. Some operations may be required in order to perform one or more processes described herein, while other operations may be more general and/or specific to a particular programming language (e.g., C, C#, C++, Java, or other suitable programming languages, etc.).

The processor 502 is operatively coupled to a communication interface 508 such that the server system 500 is capable of communicating with a remote device such as a user system 400 (shown in FIG. 5) or another server system 500. The processor 502 may also be operatively coupled to a storage device 506. The storage device 506 is any computer-operated hardware suitable for storing and/or retrieving data. In some embodiments, the storage device 506 is integrated in the server system 500. In other embodiments, the storage device 506 is external to the server system 500. For example, the server system 500 may include one or more hard disk drives as the storage device 506. In other embodiments, the storage device 506 is external to the server system 500 and may be accessed by a plurality of server systems 500. For example, the storage device 506 may include multiple storage units such as hard disks or solid-state disks in a redundant array of inexpensive disks (RAID) configuration. The storage device 506 may include a storage area network (SAN) and/or a network attached storage (NAS) system.

In some embodiments, the processor 502 is operatively coupled to the storage device 506 via a storage interface 510. The storage interface 510 is any component capable of providing the processor 502 with access to the storage device 506. The storage interface 510 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing the processor 502 with access to the storage device 506.

The memory area 504 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are exemplary only, and are thus not limiting as to the types of memory usable for storage of a computer program.

As will be appreciated based on the foregoing specification, the above-described embodiments of the disclosure may be implemented using computer programming or engineering techniques including computer software, firmware, hardware or any combination or subset thereof, wherein the technical effect is to enable the provision of one or more auxiliary medications and/or complementary foods during the dispensing of a prescription for a prescription medication, such as at a pharmacy location. Any such resulting program, having computer-readable code means, may be embodied or provided within one or more computer-readable media, thereby making a computer program product, (i.e., an article of manufacture), according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps,” or code) include machine instructions for a programmable processor and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

In an example embodiment, the system is executed on a single computer system, without requiring a connection to a server computer. In a further embodiment, the system is being run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). The application is flexible and designed to run in various different environments without compromising any major functionality. In some embodiments, the system includes multiple components distributed among a plurality of computing devices. One or more components may be in the form of computer-executable instructions embodied in a computer-readable medium. The systems and processes are not limited to the specific embodiments described herein. In addition, components of each system and each process can be practiced independently and separate from other components and processes described herein. Each component and process can also be used in combination with other assembly packages and processes.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are example only and are thus not intended to limit in any way the definition and/or meaning of the term “processor.” As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are example only and are thus not limiting as to the types of memory usable for storage of a computer program.

As used herein, an element or step recited in the singular and proceeded with the word “a” or “an” should be understood as not excluding plural elements or steps, unless such exclusion is explicitly recited. Furthermore, references to “example embodiment” or “one embodiment” of the present disclosure are not intended to be interpreted as excluding the existence of additional embodiments that also incorporate the recited features.

Disjunctive language such as the phrase “at least one of X, Y, or Z,” unless specifically stated otherwise, is generally intended, within the context presented, to disclose that an item, term, etc. may be either X, Y, or Z, or any combination thereof (e.g., X, Y, and/or Z). Likewise, conjunctive language such as the phrase “at least one of X, Y, and Z,” unless specifically stated otherwise, is generally intended, within the context presented, to disclose at least one of X, at least one of Y, and at least one of Z.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A computing device for automated provision of complementary food during prescription processing, the computing device comprising a processor in communication with a memory, the processor programmed to:
process a prescription for a first medication, the prescription including an identifier of the first medication;
detect the identifier of the first medication, from the prescription, in a stored list of flagged medications;
in response to the detecting, generate complementary food provision instructions; and
transmit the complementary food provision instructions to a pharmacy computing device of a pharmacy at which the prescription is being dispensed, the complementary food provision instructions causing the pharmacy computing device to display the complementary food provision instructions for provision of a complementary food to a patient along with the first medication.

2. The computing device of claim 1, wherein the pharmacy computing device includes a pharmacy point-of-sale device, and wherein the complementary food provision instructions cause the pharmacy point-of-sale device to initiate printing of an instructional message related to the provision of the complementary food.

3. The computing device of claim 2, wherein the complementary food provision instructions further cause the pharmacy point-of-sale device to initiate printing or electronic transmission of a promotion associated with the complementary food.

4. The computing device of claim 1, wherein the pharmacy computing device includes a pharmacy point-of-sale device, and wherein the complementary food provision instructions cause the pharmacy point-of-sale device to display an instructional message related to the provision of the complementary food on a display of the pharmacy point-of-sale device.

5. The computing device of claim 4, wherein the complementary food provision instructions further cause the pharmacy point-of-sale device to initiate printing or electronic transmission of a promotion associated with the complementary food.

6. The computing device of claim 1, wherein the prescription further includes a date at which the prescription was generated by a prescriber, and wherein the processor is further programmed to:
parse the prescription for the date;
compare the date of the prescription to a current date; and
only transmit the complementary food provision instructions when the current date is within a threshold date range relative to the date of the prescription including subsequent refills or some other interval.

7. The computing device of claim 1, wherein the processor is further programmed to:
determine whether the complementary food was offered to the patient;
determine whether the complementary food was accepted by the patient; and
when the complementary food was offered and accepted, generate an acceptance record identifying the prescription and the complementary food.

8. The computing device of claim 1, wherein the processor is further programmed to:
generate the list of flagged medications, the list of flagged medications including a plurality of entries associating product codes of prescription medications with respective product codes of complementary foods; and
store the list of flagged medications in the memory to establish the stored list of flagged medications.

9. The computing device of claim 1, wherein the processor is further programmed to:
determine an availability of the complementary food; and
when the availability is at or below a threshold, cause display of a prompt to submit an order for the complementary food.

10. The computing device of claim 1, further comprising at least one of a printer or a display device.

11. A computer-implemented method for automated provision of complementary food during prescription processing, the method implemented using a medication provision computing device comprising a processor in communication with a memory, the method comprising:
processing a prescription for a first medication, the prescription including an identifier of the first medication;
detecting the identifier of the first medication, from the prescription, in a stored list of flagged medications;
in response to the detecting, generating complementary food provision instructions; and
transmitting the complementary food provision instructions to a pharmacy computing device of a pharmacy at which the prescription is being dispensed, the complementary food provision instructions causing the pharmacy computing device to display the complementary food provision instructions for provision of a complementary food to a patient along with the first medication.

12. A computing device for automated provision of complementary food using a supplementary application, the computing device comprising a processor in communication with a memory, the processor programmed to:
store a health profile for a first user of a supplementary application;
analyze the health profile for the first user to detect a first condition including an identifier for the first condition;
detect the identifier of the first condition in a stored list of flagged conditions;
in response to the detecting, generate complementary food provision instructions; and
transmit the complementary food provision instructions to be displayed to the first user via the supplementary application, wherein the complementary food instructions cause a user computer device to display the complementary food provision instructions for provision of a complementary food to the first user.

13. The computing device of claim 12, wherein the processor is further programmed to display, via the supplemental application, an instructional message related to the provision of the complementary food.

14. The computing device of claim 12, wherein the complementary food provision instructions further cause the supplemental application to display a promotion associated with the complementary food.

15. The computing device of claim 12, wherein the processor is further programmed to cause the printing of a promotion associated with the complementary food to be sent to the first user.

16. The computing device of claim 12, wherein the processor is further programmed to generate an electronic transmission of a promotion associated with the complementary food to be transmitted to the first user.

17. The computing device of claim 12, wherein the processor is further programmed to:
determine whether the complementary food instructions were provided to the first user;
determine whether the first user purchased the complementary food; and
when the complementary food was provided and purchased, generate an acceptance record identifying the complementary food.

18. The computing device of claim 12, wherein the processor is further programmed to:
generate the list of flagged conditions, the list of flagged conditions including a plurality of entries associating product codes of respective product codes of complementary foods; and
store the list of flagged conditions in the memory to establish the stored list of flagged conditions.

19. The computing device of claim 12, wherein the processor is further programmed to:
determine an availability of the complementary food; and
when the availability is at or below a threshold, cause display of a prompt to submit an order for the complementary food.

20. The computing device of claim 12, wherein the supplemental application is a loyalty rewards application associated with at least one of a grocery store and a pharmacy.

21. The computing device of claim 12, wherein the processor is further programmed to:
receive a plurality of health information from the first user; and
generate the health profile for the first user using the plurality of health information from the first user.
